(19) United States
(12) Patent Application Publication
(10) Pub. No.: US 2003/0023132 A1
(43) Pub. Date: Jan. 30, 2003

(54) Cyclic device for restructuring heart chamber geometry

(76) Inventors: David B. Melvin, Loveland, OH (US); William P. Santamore, Medford, NJ (US)

Correspondence Address: RATNERPRESTIA, P O BOX 980, VALLEY FORGE, PA, 19482-0980, US

(21) Appl. No.: 10/210,449

(22) Filed: Jul. 31, 2002

Related U.S. Application Data
(63) Continuation-in-part of application No. PCT/US01/17637, filed on May 31, 2001.
(60) Provisional application No. 60/208,408, filed on May 31, 2000.

(51) Int. Cl. A61N 1/362

(52) U.S. Cl. 600/16

(57) ABSTRACT

A cyclical clasp for use with the natural heart. The cyclical clasp has a plurality of members configured to be positioned adjacent the epicardial surface of the heart. The members are joined by at least one connector wherein the members are fixed in a spatial or spaced relationship relative to each other such that a portion of the heart wall is displaced inwardly during at least a portion of a cardiac cycle to reconfigure a chamber of the heart as contiguous portions of truncated ellipsoids. A cyclical force is applied to the wall of the heart chamber to reduce adverse effects of reconfiguration during diastolic filling.

### CROSS-REFERENCE TO RELATED APPLICATIONS

This application is a continuation-in-part of PCT/US01/17637 filed on May 31, 2001 which claims priority of U.S. Provisional Application No. 60/208,408 filed on May 31, 2000 and which is incorporated herein by reference.

### TECHNICAL FIELD OF THE INVENTION

The present invention relates generally to a device and method for treating maladies such as cardiomyopathies and enlarged hearts and, more specifically, to a device and method for decreasing the wall stress of a heart chamber to improve systolic ejection while minimizing adverse effects on diastolic filling.

### BACKGROUND OF THE INVENTION

The natural heart and especially the cardiac muscle tissue of the natural heart (e.g., the myocardium) can fail for various reasons to a point where the heart cannot provide sufficient circulation of blood for a body to maintain life. More specifically, the heart and its chambers can become enlarged for a variety of causes and reasons, including viral disease, idiopathic disease, valvular disease (mitral, aortic, or both), ischemic disease, Chagas' disease, and so forth. As the heart and its chambers enlarge, tension of the heart chamber walls increases and, thus, the heart must develop more wall tensile stress to generate the needed pressure for pumping blood through the circulatory system. The process of ventricular dilation is generally the result of chronic volume overload or specific damage to the myocardium.

In a normal heart that is exposed to a long-term increase in cardiac output requirements, for example, that of an athlete, there is an adaptive process of right ventricular dilation and muscle myocyte hypertrophy. In this way, the heart may fully compensate for the increased cardiac output requirements of the body. With damage to the myocardium or chronic volume overload, however, there are increased requirements put on the contracting myocardium to such a level that this compensated state is never achieved and the heart continues to dilate. The adaptive process referred to above is called ventricular remodeling.

One problem with a dilated left ventricle is that there is a significant increase in wall tension or stress. The law of Laplace can be used to estimate the myocardial wall stress from the intraventricular pressure, radius of curvature and wall thickness. This undesirable increase in wall stress occurs during both the systolic and the diastolic portions of the cardiac cycle. If increases in wall stress continue unchecked, cardiac performance continues to deteriorate. As a solution for the enlarged natural heart, attempts have been made in the past to provide a treatment to maintain circulation.

Medicines, such as vasodilators, have been used to assist in treating cardiomyopathies and ventricular remodeling. For example, digoxin can increase the contractibility of the heart, and thereby enhances emptying of the chambers during systolic pumping. On the other hand, some medicines, such as beta-blocking drugs, which decrease the size of the chamber of the heart, also decrease the contractibility of the heart. Other types of medicines, such as angiotensin-converting enzyme inhibitors (e.g., enalopril) can help reduce the tendency of the heart to dilate under the increased diastolic pressure experienced when the contractibility of the heart muscle decreases. Although pharmacological management of heart failure has been demonstrated to be partially effective in preventing and reversing the disease process, the long term hemodynamic unloading of the heart by these drugs is often not possible as these medicines have severe side effects, such as excessive lowering of blood pressure.

Besides medical therapy an alternative procedure is to transplant a heart from another human or animal into a patient. Heart transplantation is the most definitive treatment in patients with end stage disease. The transplant procedure requires removing an existing organ (i.e., the natural heart) for substitution with another organ (i.e., another natural heart) from another human or, potentially, from an animal. Before replacing an existing organ with another, the substitute organ must be “matched” to the recipient, which can be, at best, difficult and time consuming to accomplish. Further, even if the transplanted organ matches the recipient, a risk exists that the recipient's body will reject the transplanted organ and attack it as a foreign object. Still further, the number of potential donor hearts is far less than the number of patients in need of a transplant. Although use of animal hearts would lessen the problem of fewer donors than recipients, there is an enhanced concern with rejection of the animal heart. For these reasons and the significant cost associated with heart transplantation it remains the treatment of last resort for congestive heart failure patients.

Another approach is to replace the existing natural heart in a patient either physically with an artificial heart or functionally with a ventricular assist device. In using either artificial hearts or ventricular assist devices, however, the materials adapted for the interior lining of the chambers of an artificial heart or ventricular assist device are in direct contact with the circulating blood, which can enhance undesirable clotting of the blood, build up of calcium, or otherwise inhibit the normal function of the blood. Hence, thromboembolism and hemolysis could occur with greater ease. In addition, the lining of an artificial heart or a ventricular assist device can crack, which inhibits performance even if the crack is at a microscopic level. Moreover, these devices must be powered by a source which can be cumbersome and may be external to the body. Such drawbacks in addition to their cost have limited use of these devices to applications having too brief an effective time period to provide a real lasting benefit.

In an effort to use the existing natural heart of a patient, other attempts have been made to reduce wall tension of the heart by removing a portion of the heart wall, such as a portion of the left ventricle in a partial left ventriculectomy procedure (the Batista procedure). The rationale for this invasive surgical treatment was Laplace's law. A wedge-shaped portion of the ventricular muscle has been removed, which extends from the apex to the base of the heart. By reducing the chamber's volume, and thus its radius, the tension of the chamber's wall is reduced as well according to the law of Laplace. There are several drawbacks, however, with such a procedure. First, a valve (i.e., the mitral valve) may need to be repaired or replaced depending on the amount of cardiac muscle tissue to be removed. Second, the procedure is invasive and traumatic to the patient. As such, blood loss and bleeding can be substantial during and after the procedure. Moreover, as can be appreciated by those skilled in the industry, the procedure is not reversible. Although the Batista procedure reduces wall stress and provides short term beneficial effects on systolic function, it also results in adverse effects on diastolic function.

Another device developed for use with an existing heart for sustaining the circulatory function of a living being and the pumping action of the natural heart is an external bypass system, such as a cardiopulmonary (heart-lung) machine. Typically, bypass systems of this type are complex and large and, therefore, are limited to short-term use in an operating room during surgery or to maintaining the circulation of a patient while awaiting receipt of a transplant heart. The size and complexity effectively prohibit use of bypass systems as a long-term solution; they are rarely even portable devices. Furthermore, long-term use of these systems can damage the blood cells and blood-borne products, resulting in post-surgical complications such as bleeding, thromboembolism and increased risk of infection.

Yet another device developed for use with an existing heart for sustaining the circulatory function of a living being and the pumping action of the natural heart is disclosed in U.S. Pat. No. 6,190,408 issued to Dr. David B. Melvin. A clasp with members configured to be positioned adjacent the epicardial surface of the heart, restraining portions of the wall of a chamber and reconfiguring the chamber of the heart. This reconfiguration reduces wall stress, improving systolic ejection. Although this device geometrically reshapes the natural heart, reduces wall stress and enhances systolic performance, is not known to directly and immediately reduce adverse effects during diastolic filling of the natural heart.

Yet other restraining devices, intended for use with an existing heart to either reduce or constrain the radius of curvature in attempts to reduce or control wall stress by favorably impacting ventricular function are reflected in U.S. Pat. No. 6,050,936 issued to C. J. Schweich; U.S. Pat. No. 5,702,343 issued to C. A. Alferness and U.S. Pat. No. 5,800,528 issued to D. M. Lederman. Although some of these devices have been described in the literature they have not been expressed in terms of the end-systolic and end-diastolic pressure-volume relationships. While at least some of these devices may provide positive effects on systolic function, none of them provide or suggest minimizing potentially adverse effects on diastolic filling of the natural heart.

As can be seen, currently available treatments, procedures, medicines, and devices for treating end-stage cardiomyopathies have a number of shortcomings that contribute to the complexity of the procedure or device. Some of the current procedures and therapies are extremely invasive, and may provide a benefit for only a brief period of time. They may also have undesirable side effects which can hamper the heart's effectiveness. There exists a need in the industry for a device and procedure that can interact with the existing heart to provide a practical, long-term device and procedure to reduce wall tension of the heart, and thus improve its pumping efficiency while minimizing adverse effects on diastolic filling.

### SUMMARY OF THE INVENTION

To meet these and other needs, it is the object of the present invention to provide a device and method for treating a natural heart that addresses and overcomes the problems and shortcomings mentioned above in the cardiac surgical and cardiology arts. To achieve this and other objects, and in view of its purposes, the present invention provides a cyclical clasp for treating a natural heart that has a plurality of members configured to press inwardly on the walls of a chamber of the heart, reconfiguring the chamber and reducing wall stress during at least one portion of a cardiac cycle. The device is further configured to reduce adverse effects on expansion of the chamber during a second portion of a cardiac cycle. In one embodiment, the device may further comprise an energy-transfer mechanism that stores energy from the natural heart during one portion of the cardiac cycle and releases the stored energy during a second portion of the cardiac cycle.

In one embodiment of the present invention, the energy-transfer mechanism is a spring, which releases energy to reduce the distance between members adapted to reconfigure a chamber of the heart during at least a portion of a cardiac cycle. While the members are in a closely spaced relationship, wall stress is reduced which can improve systolic function. The spring force is overcome by wall tension during late systole and/or isovolumic relaxation. The force exerted by the chamber walls due to the tension forces the members apart, increasing the distance between the members to a distantly spaced relationship and imparting energy into the spring. While the members are in a distantly spaced relationship, minimal restraint is applied to the heart chamber and the adverse effects on diastolic function are reduced. During late diastole and/or isovolumic contraction, the reduced wall tension is overcome by the spring, forcing the members closer together, back into the closely spaced relationship. The device further comprises a locking mechanism to maintain the members in a desired spaced relationship during at least one portion of a cardiac cycle.

In another embodiment of the present invention, the energy transfer mechanism is a pressure-transfer mechanism that applies a cyclical outward force to the endocardial surface of a chamber wall. The pressure transfer mechanism is combined with a clasp to reconfigure a chamber of the heart, reducing wall stress and improving systolic function while cyclical outward force from the pressure-transfer mechanism enhances diastolic function to eliminate or reduce any net adverse effects on diastolic function. The pressure-transfer mechanism absorbs and stores energy when it is deflected or compressed during systolic ejection. The stored energy is then released during diastolic filling to enhance the diastolic filling of the chamber.

In use, the present invention can reduce the wall tension on one of the chambers of the heart during at least one phase of the cardiac cycle. A clasp is affixed to the heart so as to provide the chamber of the heart as at least two contiguous communicating regions, such as sections of truncated ellipsoids, which have a lesser minimum radii than the chamber before restructuring. As such, the clasp displaces at least two portions of the chamber wall inwardly from the unrestricted position during at least a portion of a cardiac cycle. The clasp is further configured to apply cyclical forces to a chamber wall of a natural heart to reduce the adverse effects of the clasp on diastolic filling. This can be accomplished by incorporating an energy transfer mechanism into the clasp itself, so that the clasp restructures the chamber during a portion of a cardiac cycle and the chamber is not restructured during another portion of the cardiac cycle. Alternatively, a pressure-transfer mechanism can act on the endocardial surface of the chamber to absorb energy during one portion of the cardiac cycle and release that energy during another portion of the cardiac cycle.

It is to be understood that both the foregoing general description and the following detailed description are exemplary, but are not restrictive, of the invention.

### BRIEF DESCRIPTION OF THE DRAWING

The invention is best understood from the following detailed description when read in connection with the accompanying drawing. It is emphasized that, according to common practice, the various features of the drawing are not to scale. On the contrary, the dimensions of the various features are arbitrarily expanded or reduced for clarity. Included in the drawing are the following figures:

FIG. 1 is a partial front anterior view of an exemplary natural heart;

FIG. 2 is a vertical cross-sectional view of an exemplary natural heart and blood vessels leading to and from the natural heart;

FIG. 3 is a horizontal cross-sectional (short axis) view of an unrestrained left ventricle of the natural heart;

FIG. 4 is a perspective view of a device made in accordance with the present invention and positioned on the left ventricle;

FIG. 5 is a cross-sectional view taken along line 5-5 in FIG. 4 showing a device restraining a chamber of a natural heart according to one embodiment of the present invention;

FIG. 6 is one of two restraining bars of a clasp in accordance with one embodiment of the present invention in which members are in a distantly spaced relationship with each other;

FIG. 7 is one of two restraining bars of a clasp in accordance with the present invention in which members are in a closely spaced relationship with each other;

FIG. 8 is a partial horizontal cross-sectional view of a natural heart showing a chamber which is essentially unrestricted by a device with members in a distantly spaced relationship in accordance with one embodiment of the present invention with restraining bars in a distantly spaced relationship;

FIG. 9A is a diagram illustrating Aortic Pressure (Ao), the left ventricular pressure, and volume versus time for a normal heart;

FIG. 9B is a diagram illustrating the left ventricular pressure-volume relationship for the normal heart of FIG. 9A;

FIG. 10 is a diagram of a control system for a cyclical clasp according to one embodiment of the present invention;

FIG. 11 illustrates an embodiment of an energy transfer mechanism and locking mechanism disposed on a segment of a clasp according to an embodiment of the present invention;

FIG. 12 is a time plot of the operation of a clasp according to an embodiment of the present invention showing from top to bottom: an EKG, left ventricular pressure (LVP), cable/locking mechanism position, and heart chamber/restraining bar cross section, all on the same horizontal time axis;

FIG. 13 is a comparative diagram of left ventricle pressure (LVP) versus volume (LVV) for a natural canine heart showing experimentally obtained end systole pressure (ESP) and end diastole pressure (EDP) curves and pressure-volume (PV) cycles with a fixed reconfiguration device and with no device;

FIG. 14 is a left ventricle pressure versus volume diagram for the natural canine heart of FIG. 13 showing a calculated PV cycle for a cyclical clasp according to one embodiment of the present invention;

FIG. 15 is a cyclical clasp according to an alternative embodiment of the present invention;

FIG. 16 is a sectional view of a hinge and locking mechanism for use with the embodiment of FIG. 15;

FIG. 17 is a sectional view of an alternate hinge and locking mechanism for use with the embodiment of FIG. 15 with the locking mechanism locked;

FIG. 18 is a sectional view of an alternate hinge and locking mechanism for use with the embodiment of FIG. 15 with the locking mechanism unlocked;

FIG. 19 is a pressure-transfer mechanism configured to be positioned inside a chamber of a natural heart such that it absorbs energy in a first state during systolic ejection and releases the stored energy to enhance diastolic filling in a second state;

FIG. 20 is a partial horizontal cross-sectional view of a natural heart showing a chamber which is reconfigured to reduce wall stress by restraining bars in a fixed closely spaced relationship and enhanced during diastolic filling by the pressure transfer-mechanism of FIG. 19;

FIG. 21 is a partial horizontal cross-sectional view of a natural heart showing a chamber which is reconfigured to reduce wall stress by restraining bars in a fixed closely spaced relationship and enhanced during diastolic filling by an alternative pressure-transfer mechanism;

FIG. 22 is a partial longitudinal section of a natural heart showing a chamber which is reconfigured to reduce wall stress by restraining bars in a fixed closely spaced relationship and enhanced during diastolic filling by another alternative pressure-transfer mechanism;

FIG. 23 is a partial longitudinal section of a natural heart showing a chamber which is reconfigured to reduce wall stress by restraining bars (omitted for clarity) in a fixed closely spaced relationship and enhanced during diastolic filling by yet another alternative pressure-transfer mechanism; and

FIG. 24 is a partial longitudinal section of a natural heart showing a chamber which is reconfigured to reduce wall stress by restraining bars in a fixed closely spaced relationship and enhanced during diastolic filling by still another alternative pressure-transfer mechanism.

### DETAILED DESCRIPTION OF THE INVENTION

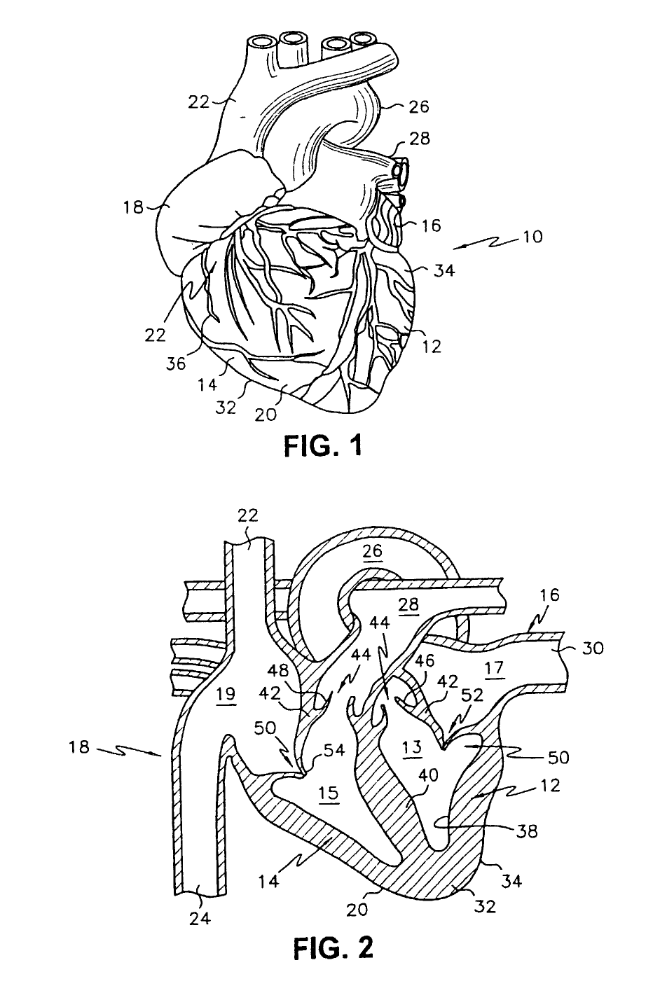

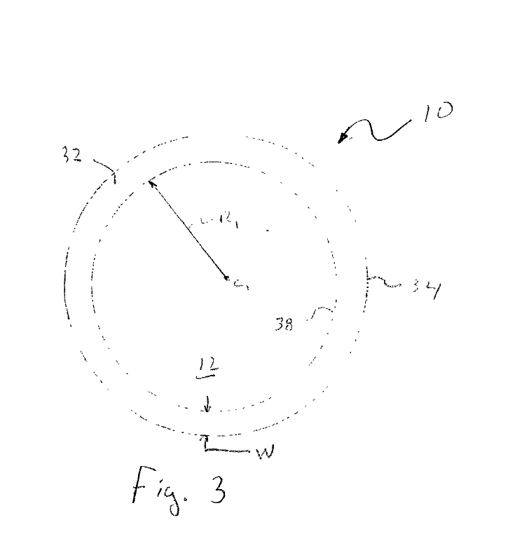

Referring now to the drawing, in which like reference numbers refer to like elements throughout, FIGS. 1 and 2 show an exemplary natural heart 10. The heart 10 has an apical portion 20 (i.e., a lower portion) comprising two chambers, namely a left ventricle 12 and a right ventricle 14, which function primarily to supply the main force that propels blood through the circulatory system, namely the pulmonary circulatory system, which propels blood to and from the lungs, and the peripheral circulatory system, which propels blood through the remainder of the body. The heart 10 also includes an upper portion having two chambers, a left atrium 16 and a right atrium 18, which primarily serve as an entryway to the left and right ventricles 12 and 14, respectively, and assist in moving blood into the left and right ventricles 12 and 14. The interventricular wall 40 of the cardiac tissue 32 separates the left and right ventricles 12 and 14, and the atrioventricular wall 42 of the cardiac tissue 32 separates the lower ventricular region from the upper atrium region.

Generally, the left and right ventricles 12 and 14, respectively, each has a cavity 13 and 15, respectively, that is in fluid communication with the cavities 17 and 19, respectively, of the atria (e.g., 16 and 18) through an atrioventricular valve 50 (both atrioventricular valves 50 are illustrated as closed in FIG. 2). More specifically, the left ventricle cavity 13 is in fluid communication with the left atrium cavity 17 through the mitral valve 52, while the right ventricle cavity 15 is in fluid communication with the right atrium cavity 19 through the tricuspid valve 54.

Generally, the cavities of the ventricles (e.g., 13 and 15) are each in fluid communication with the circulatory system (i.e., the pulmonary and peripheral circulatory systems) through a semilunar valve 44 (both semilunar valves 44 are illustrated as opened in FIG. 2). More specifically, the left ventricle cavity 13 is in fluid communication with the aorta 26 of the peripheral circulatory system through the aortic valve 46, while the right ventricle cavity 15 is in fluid communication with the pulmonary artery 28 of the pulmonary circulatory system through the pulmonic valve 48.

Blood is returned to the heart 10 through the atria (e.g., 16 and 18). More specifically, the superior vena cava 22 and inferior vena cava 24 are in fluid communication and deliver blood, as it returns from the peripheral circulatory system, to the right atrium 18 and its cavity 19. The pulmonary vein 30 is in fluid communication and delivers blood, as it returns from the pulmonary circulatory system, to the left atrium 16 and its cavity 17.

The heart 10 is enclosed in the thoracic cavity within a double-walled sac commonly referred to as the pericardium. Its inner layer is the visceral pericardium or epicardium, and its outer layer is the parietal pericardium. The structure of the heart 10 is generally made up, among other materials, of cardiac muscle or tissue 32 which has an exterior surface commonly known as the epicardial surface 34 and an interior surface, or endocardial surface 38, that generally defines the cavities (e.g., ventricular cavities 13 and 15, respectively, and atrial cavities 17 and 19, respectively). Coronary arteries 36 on the epicardial surface 34 of the heart 10 provide blood and nourishment (e.g., oxygen) to the heart 10 and its cardiac tissue 32.

By way of a non-limiting example, the present invention will be discussed in terms of embodiments that are used to primarily assist in the restructure, reconfiguration, or operation of the left ventricle 12 of the natural heart 10. The present invention can also be used, however, to assist in the restructure, reconfiguration, or operation of other portions of the natural heart 10. Such other portions include either atria 16, 18 and the right ventricle 14 of the natural heart 10.

Turning now to FIG. 3, the chambers of the heart 10, including the left ventricle 12, are generally shaped as a hollow truncated ellipsoid. The ellipsoid has, at any circular cross-section perpendicular to its long axis, a center of curvature “C₁” and a radius “R₁” extending from center point C₁ to the endocardial surface 38. The cardiac tissue 32 of the heart 10 has a thickness “w,” which is generally the distance between the epicardial surface 34 and the endocardial surface 38. FIG. 3 illustrates a cross-section of the left ventricle of a heart 10 in its natural, unrestricted position.

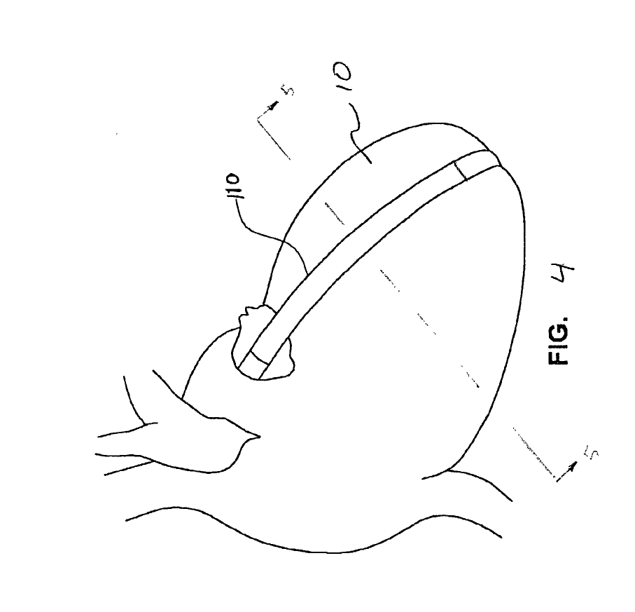

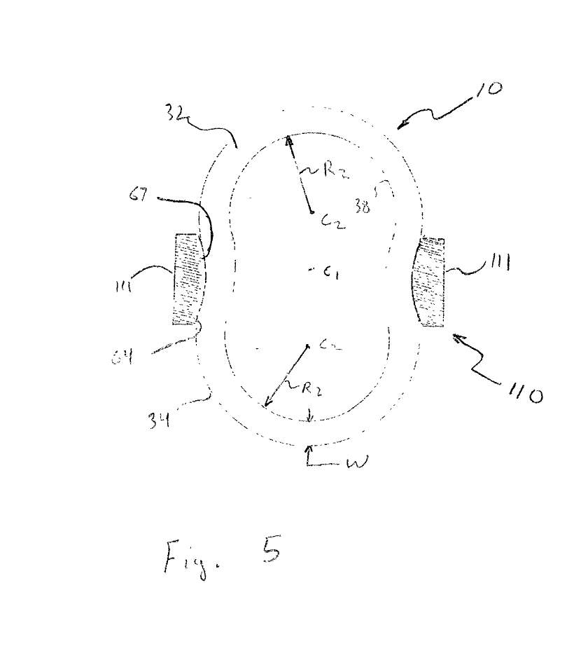

The clasp 110 of the present invention exemplified in FIGS. 4 and 5 preferably is configured and positioned relative to the natural heart 10 to displace at least two portions of the cardiac tissue 32 inwardly (see, e.g., FIG. 5) from the unrestricted position exemplified in FIG. 3. By displacing portions of the cardiac tissue 32 inwardly, the shape of the chamber (e.g., the left ventricle 12) of the heart 10 is generally restructured or reconfigured from a generally hollow truncated ellipsoid (see, e.g., FIG. 3) to a chamber generally shaped as having at least two continuous communicating portions of truncated ellipsoids (see, e.g., FIG. 5). In generally reconfiguring or restructuring the heart 10, each of the truncated ellipsoids has an adjusted radius “R₂,” which is preferably shorter than radius “R₁,” measured with reference to an adjusted center of curvature “C₂.”

The clasp 110 preferably includes at least two members 111 (e.g., restraining bars) to assist in restraining or restructuring the left ventricle 12. The restraining bars 111 are preferably spaced about 180 degrees apart (in the cross-sectional view) adjacent or on the epicardial surface 34 so as to restructure or reconfigure the left ventricle 12 as having the shape of at least two continuous communicating portions of truncated ellipsoids. One of the members 111 can be configured to be positioned adjacent the anterolateral surface of a chamber (e.g., the left ventricle 12) and the other member 111 can be configured to be positioned adjacent the posteromedial surface of a chamber (e.g., the left ventricle 12).

Each member 111 includes a contact or an inner surface 64 that is preferably curved convex outwardly in a longitudinal plane (see, e.g., FIG. 5) and curved convex inwardly in the transverse plane (see, e.g., FIG. 4) so that member 111 is configured to be positioned adjacent or on the epicardial surface 34 whereby intimate contact can be established and maintained, even during the contraction or beating of the heart 10. The inner surface 64 of member 111 is configured so that it is tangent to the portion of the epicardial surface 32 where contact is made and so that the cardiac tissue 32 is altered or displaced in a transverse plane, from its unrestructured inwardly convex shape (see, e.g., FIG. 3) to its restructured concave shape (see, e.g., FIG. 5). The inner surface 64 can be provided as a smooth curved surface having lateral portions 67 such that the epicardial surface 34 may slide along the member 111 during contraction and expansion of the heart 10.

Members 111 are each preferably made of a light weight, generally rigid material that has a low bending strain under expected levels of stress. This material provides sufficient wear resistance in use while the heart 10 beats and maintains its desired shape in use adjacent the heart 10. Illustrative examples of materials which may be employed as members 111 include any biocompatible materials such as metals, including titanium or stainless steel, suitable polymers including polyacetal or liquid crystal polymers (LCPs), and ceramic materials.

Members 111 can be any desired shape, and can vary according to anatomy and the desired application. Preferably, members 111 have rounded edges and are generally rectangular-shaped with the length extending in the transverse plane or along the longer axis of the chamber (i.e., extending between the basal portion near the atrioventricular groove (not shown) and apical portion 20 of the heart 10). In a preferred embodiment, members 111 contact the epicardial surface of a natural heart over a length that varies from about 50% to about 100% of the vertical long axis of the chamber (e.g., 12), approximately from about 4 to about 12 cm, and preferably over a length of about 80% of the vertical long axis of the chamber (e.g., 12). Moreover, the member 111 can have a thickness that varies from about 1 mm to about 10 mm, depending on the modulus and strength of the material chosen. When metal is used for member 111, the member 111 can preferably have a thickness of about 1 mm, and when a high strength polymer is used for member 111, the member 111 can have a thickness that varies from about 6 mm to about 8 mm.

The member 111 can be an assembly including components comprising different materials so that desired properties can be optimized for a specific purpose. For example, the desired longitudinal rigidity can be obtained with a metal bar while the desired tangential contact with the epicardial surface can be obtained with a pad that is intrinsically torsionally or longitudinally flexible riding on the rigid metal bar. The transversely flexible pad can comprise a series of rigid elements that can independently pivot on the rigid bar. These elements can, for example, be embedded on a suitable low durometer elastomer or alternatively threaded onto the bar.

The clasp 110 is passive in that it does not actuate or pump the heart 10. Rather, the clasp 110 displaces and holds portions of the cardiac tissue 32 in a generally predetermined fixed position as the heart 10 contracts (e.g., beats) and pumps blood through its chambers and through the body's circulatory system. Then, as the chambers of the heart fill with blood, the clasp 110 reduces the adverse effects on filling caused by restraining the heart. In a first exemplary embodiment of the present invention, the clasp 110 reduces these adverse effects by repositioning the restraining bars 111 that displace portions of the cardiac tissue 32 to reduce impediment to filling. Alternatively, a clasp may include a pressure transfer mechanism that applies an outward force on the walls of a heart chamber to enhance filling as illustrated in FIGS. 19-24 and described below.

A fixation device (not shown) can be configured to maintain contact between the members 111 and a specific surface of the heart chamber. During heart contractions, the clasp 110 can become dislocated from its desired position. Accordingly, a fixation device may be incorporated in the clasp 110 to maintain the clasp 110 in contact with the specific desired location of the surface of the chamber wall. “Fixation devices” (as the term is used in this document) may refer to a mechanical fastener such as a pin or screw that penetrates the surface of the chamber wall or it may refer to a strap or tether which ties the clasp 110 to a structure of the heart or other means as will be apparent to those skilled in the art.

A. Cyclical Clasp

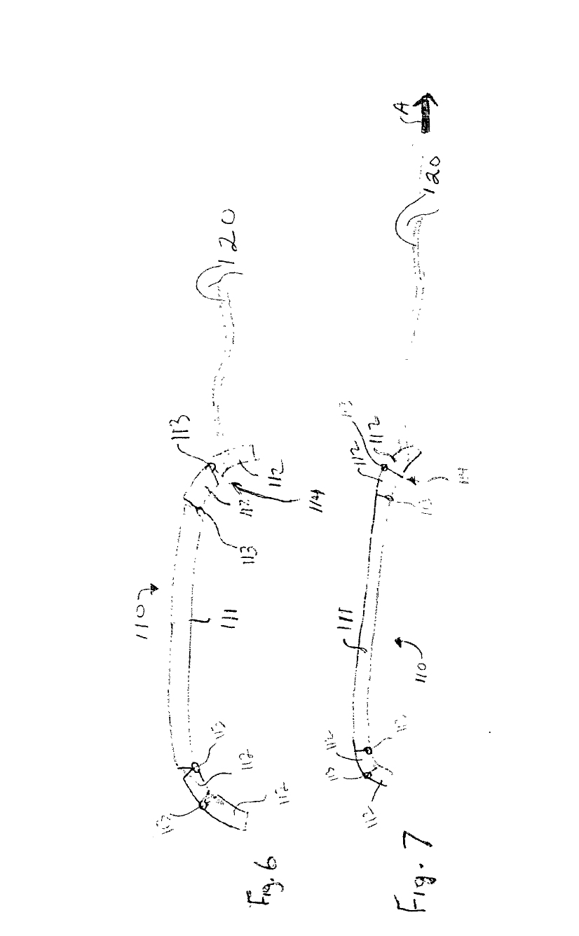

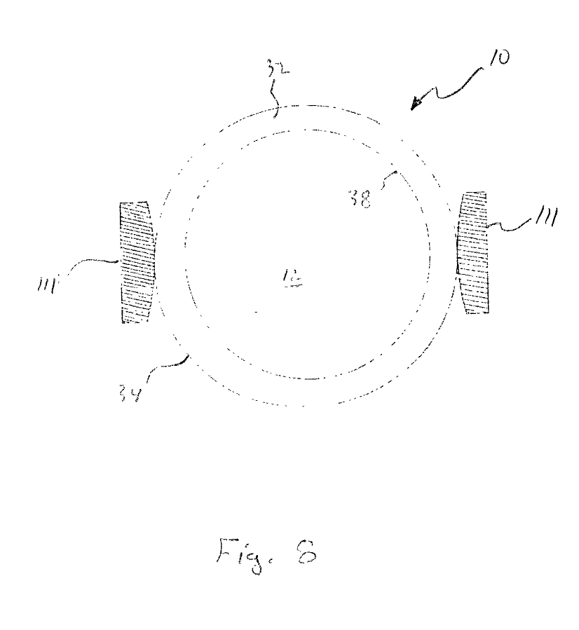

Referring now to FIG. 6, in one group of embodiments of the invention, the clasp 110 comprises a plurality of members (e.g., restraining bars) 111 connected by a connector comprising hinged connecting segments 112. Each restraining bar 111 is configured to be positioned adjacent the epicardial surface 34 of a natural heart 10. Connecting segments 112 are joined at each end to a restraining bar 111 or another connecting segment 112 by a hinge 113, forming hinged joints 114. When the hinged joints 114 are closed, as shown in FIG. 7, the restraining bars 111 are fixed in a first or closely spaced relationship to each other. When the hinged joints 114 are open, as shown in FIG. 6, the restraining bars 111 are allowed to move to a second or distantly spaced relationship, in which the distance between restraining bars 111 is greater than in the first spaced relationship. As used in this document, the term “closely spaced” means that the restraining bars 111 are spaced such that they apply a certain inward pressure to displace or indent the underlying portions of the epicardial surface 34, and the term “distantly spaced” means that the restraining bars 111 are spaced such that they apply a minimal or preferably essentially no inward pressure and the underlying portions of the epicardial surface 34 are displaced or indented to a lesser degree, or, preferably, not at all.

A tension member 120 is disposed in a lumen (not shown) running through or adjacent the restraining bars 111 and the connecting segments 112. The tension member 120 has a variable effective length, which depends upon the magnitude of tension applied to it. When the tension member 120 is subjected to increased tension, as represented by the arrow “A” in FIG. 7, the effective length of the tension member 120 is reduced and the hinged joints 114 are fixed in a closed position. When tension is relaxed on tension member 120, the hinged joints 114 are allowed to open as shown in FIG. 6.

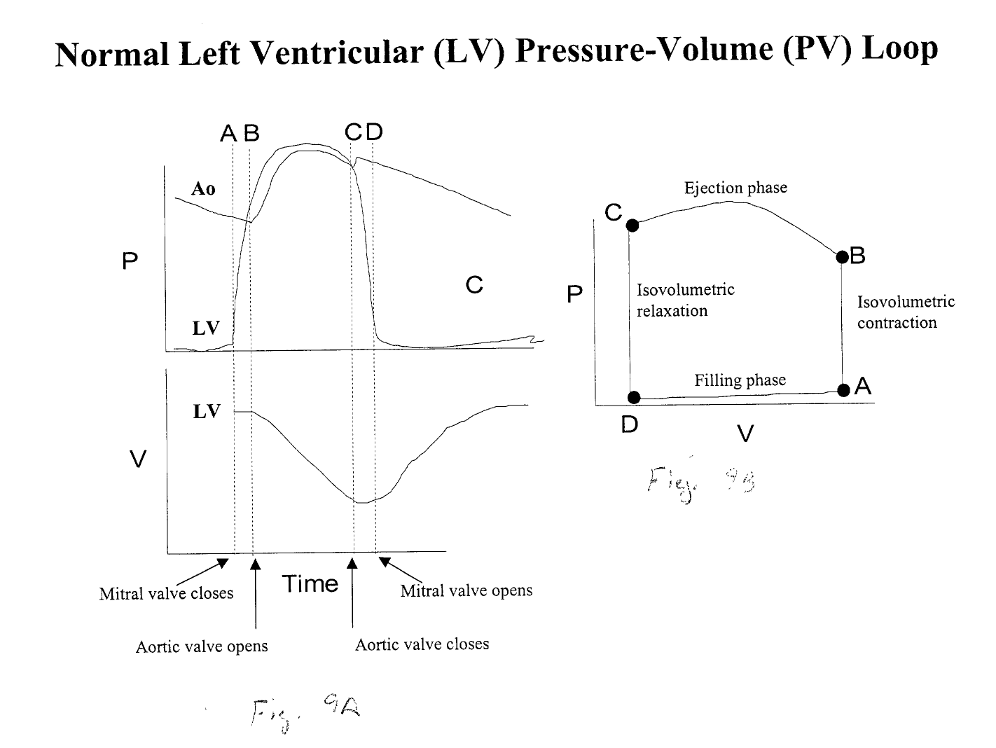

FIG. 5 shows a sectional view of the restraining bars 111 and left ventricle 12 of the heart 10 illustrating the closely spaced relationship, and FIG. 8 shows a sectional view of the restraining bars 111 and left ventricle in a distantly spaced relationship of a device according to one embodiment of the present invention. When hinged joints 114 (see FIG. 6) are closed, restraining bars 111 are in a closely spaced relationship as shown in FIG. 5, and underlying portions of the chamber wall are displaced inwardly from their unrestricted position to reconfigure left ventricle 12 as contiguous portions of truncated ellipsoids to reduce wall stress. When the hinged joints 114 are open, the restraining bars 111 are in a distantly spaced relationship as shown in FIG. 8, and the chamber walls are essentially unrestricted.

As disclosed above, the reconfiguration shown in FIG. 5 reduces the radius of the heart chamber, thereby reducing the stress in the chamber wall. This reduced stress is particularly desirable during systolic ejection when peak stress occurs, because reducing systolic stress decreases the load on the myocardial cells, leading to improved ventricular performance. Reconfiguring the left ventricle 12 during diastolic filling may not be beneficial, however, because this restraining can inhibit filling by increasing chamber stiffness (i.e., diastolic elastance).

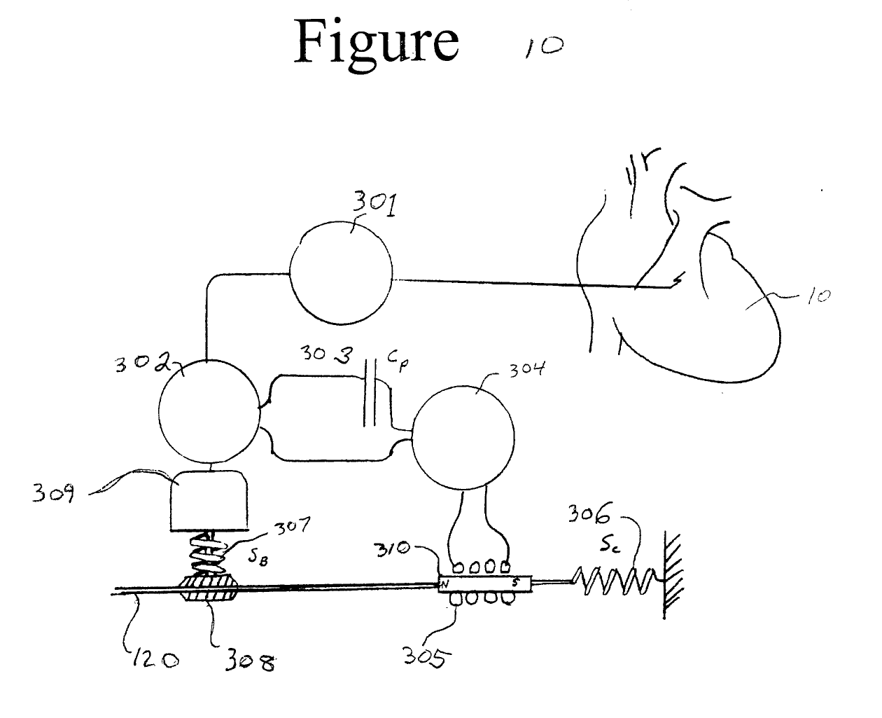

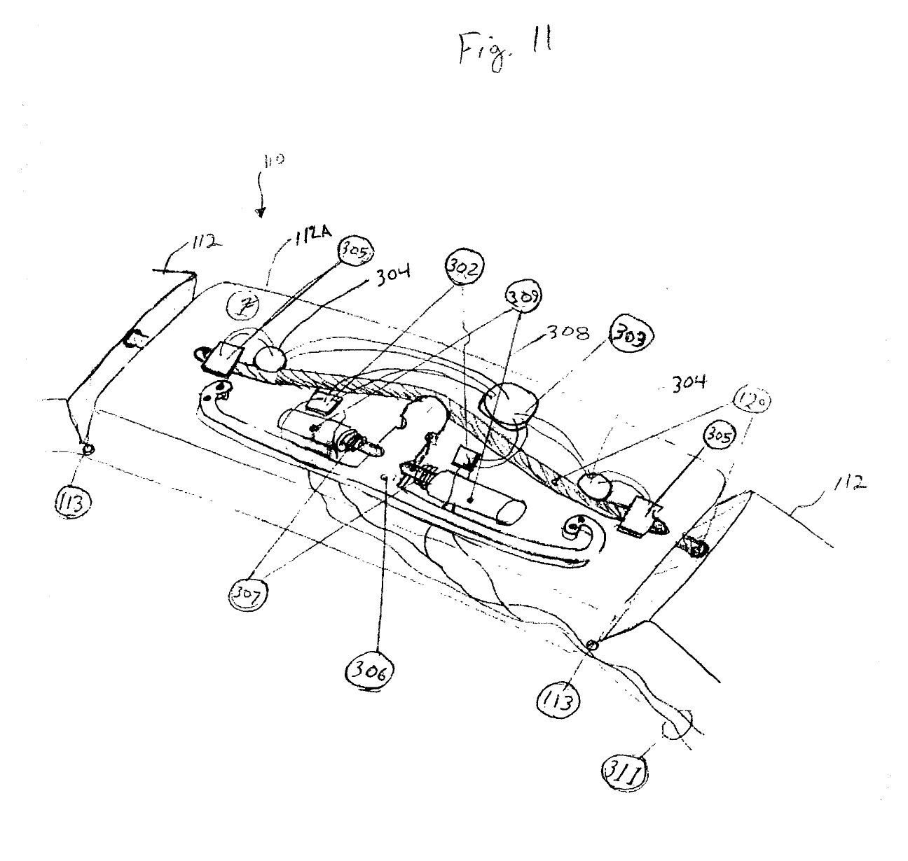

FIGS. 9A and 9B represent the normal left ventricular pressure-volume relationship or loop. FIG. 9A shows the left ventricular (LV) and aortic (Ao) pressure curves (top) and the LV volume curve (bottom) versus time for a normal, unrestricted left ventricle. FIG. 9B shows a pressure-volume (PV) loop for a normal, unrestricted left ventricle. The normal cardiac cycle progresses counterclockwise around the PV loops. At point D the mitral valve opens, initiating diastolic filling (line DA). During diastolic filing of the left ventricle (line DA), the volume increases in association with a gradual rise in pressure. When ventricular contraction commences and its pressure exceeds that of the left atrium, the mitral valve closes (point A) and isovolumic contraction of the left ventricle ensues (the aortic valve is not yet open and no blood leaves the chamber) as shown in line AB. When left ventricular pressure exceeds that in the aorta (see FIG. 9A of LV and Ao pressure versus time), the aortic valve opens (point B) and ejection begins. The volume within the left ventricular declines during systolic ejection (line BC), but left ventricular pressure remains elevated until ventricular relaxation commences. At point C the left ventricular pressure during relaxation falls below that in the aorta and the aortic valve closes, leading to isovolumic relaxation (line CD). Reopening of the mitral valve (point D) occurs when LVP falls below left artial pressure. Point A represents the end-diastolic volume and pressure, and point C is the end-systolic volume and pressure. The stroke volume is the difference between end-diastolic volume (at point A) and end-systolic volume (at point C). The stroke work is defined by the area enclosed by the pressure-volume loop.

In a cyclically adjusting clasp embodiment of the present invention, the PV cycle can be utilized to assist in repositioning restraining bars (111 in FIGS. 5 and 8). Cardiac performance can be enhanced by positioning the restraining bars in a closely spaced relationship (as shown in FIG. 5) during systolic ejection, and potentially adverse effects of the restraining bars can be reduced by positioning them in a distantly spaced relationship (as shown in FIG. 8). The chamber pressure just before and just after the end of ejection (point C) results in a relatively high wall tension despite a relatively low volume. If, at that point, the restraining bars are in a closely spaced relationship, this high wall tension will exert a strong outward force on the restraining bars. That force may be employed to displace or reposition the restraining bars to a separation distance approaching the undeformed end-systolic ventricular diameter (i.e., a distantly spaced relationship). If required, a motor or other extrinsic power source of a type known to those familiar with the art of biomechanical engineering may be used to supplement or provide energy for this action. At the same time that the restraining bars are repositioned to a distantly spaced relationship, energy can be stored by an energy transfer mechanism, such as a spring, for later inward repositioning. If the restraining bars are maintained in a distantly spaced relationship during diastolic filling, then potentially adverse effects caused by reconfiguration are reduced.

Then, near the end of diastolic filling (point A), there is a relatively low chamber pressure. This low chamber pressure results in a low wall tension such that the restraining bars can be inwardly displaced or repositioned to a closely spaced relationship with very little force, preferably no more force than was stored in the energy transfer device while repositioning the restraining bars to a distantly spaced relationship. However, this action may, if required, be supplemented by a motor or other power source, of a type known to those familiar with the art of biomechanical engineering. If the restraining bars are maintained in a closely spaced relationship during systolic ejection, then wall stress is reduced, improving ejection performance.

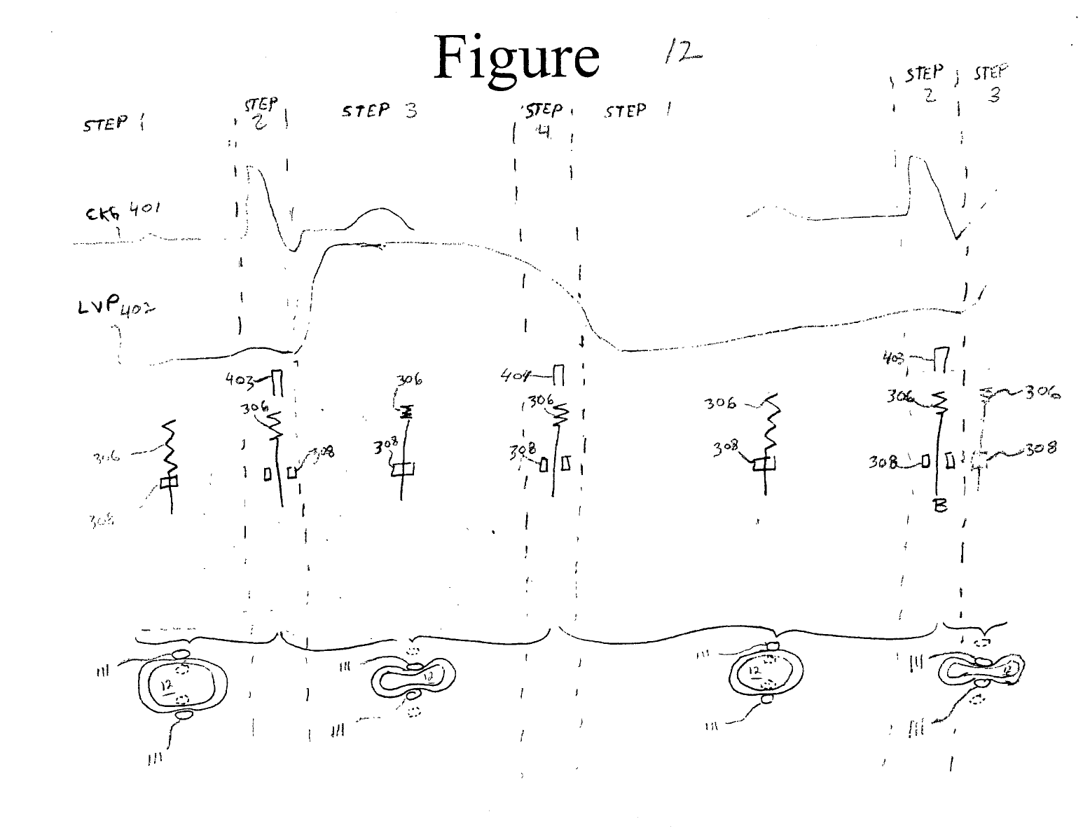

FIG. 10 illustrates a control system for a cyclical clasp 110 according to one embodiment of the present invention. An energy transfer mechanism 306 is attached to the tension member 120 providing a tension sufficient to overcome the lower chamber pressure at around point A of FIGS. 9A and 9B (late diastolic filling and/or early isovolumic contraction. The pressure is not, however, sufficient to overcome the greater chamber pressure at point C of FIGS. 9A and 9B (i.e., late systolic ejection and/or early isovolumic relaxation). Energy transfer mechanism 306 can be, for example, a spring with a spring constant (Sc) and length chosen to provide a tension to tension member 120 sufficient to overcome the pressure in the heart chamber (e.g., left ventricle 12 shown in FIGS. 5 and 8) at about point A of a cardiac cycle shown in FIGS. 9A and 9B (i.e., during late diastolic filling and/or early isovolumic contraction), thereby closing the hinged joints 114 (see FIGS. 6 and 7) and providing the closely spaced relationship of restraining bars 111 to reconfigure the chamber as shown in FIG. 5 prior to systolic ejection. Alternatively, energy transfer mechanism 306 may be an elastomeric element, a compressible fluid, or another energy transfer mechanism suitable for use in a human body and capable of transferring kinetic energy to potential energy and potential energy to kinetic energy, of the type known to those familiar with the art of biomechanical engineering. The tension provided by the chosen energy transfer mechanism 306 is not sufficient, however, to overcome the pressure in the heart chamber at about point C of a cardiac cycle shown in FIGS. 9A and 9B. Therefore, during late systolic ejection and/or early isovolumic relaxation, the pressure in the heart chamber overcomes the tension provided to tension member 120 by energy transfer mechanism 306 opening hinged joints 114 and repositioning the members 111 to a distantly spaced relationship so that the chamber is essentially unrestricted as shown in FIG. 8 prior to diastolic filling.

A locking mechanism (i.e., brake) 308 is applied to tension member 120 to maintain the closely spaced relationship as chamber pressure increases during systolic ejection. Without locking mechanism 308, the increasing chamber pressure would overcome the tension provided by energy transfer mechanism 306 and displacement (i.e., reconfiguration) would be lost. As shown in FIG. 10, locking mechanism 308 can be applied, for example, by a locking spring 307 with a spring constant (SB), acting against the solenoid 309 when the solenoid 309 is de-energized. It should be noted that failure of the locking mechanism or control system would result in the locking mechanism remaining locked and the clasp functioning as a fixed (non-cyclical) clasp.

The solenoid 309 is energized by a relay switch 302 following a first control signal generated by a programmable cardiac-sensing electronic circuit (e.g., one or more DDD pacemakers) 301. Alternatively, relay switch 302 may be triggered by a pressure sensor or other device to coordinate energizing the solenoid with the cardiac cycle or other physiologic signal. The cardiac-sensing circuit 301 is programmed to provide a first control signal to the solenoid 309 shortly before systolic ejection (late diastole or early isovolumic contraction), such as when an R-wave is generated by the heart 10, and a second control signal near the end of systolic ejection or the beginning of isovolumic relaxation, for example, following a delay of about 0.4 seconds after an R-wave is generated by the heart 10. The first control signal, generated shortly before systolic ejection triggers the solenoid 309, allowing the energy transfer mechanism 306 to apply tension to the tension member 120. The tension in tension member 120 overcomes the wall stress of the heart chamber, moving restraining bars 111 (not shown in FIG. 10, but illustrated on the timeline of FIG. 12) to a closely spaced relationship and reconfiguring the heart chamber. Shortly after this reconfiguration, the solenoid 309 is deactivated, thereby locking the restraining bars 111 in this position. The second control signal is generated during late systole and/or isovolumic relaxation, again triggering the solenoid 309, allowing the chamber pressure (which is now greater than the tension applied to the tension member 120 by the energy transfer mechanism 306) to move the restraining bars 111 to a remotely spaced relationship, with storing of energy. Shortly thereafter, the solenoid 309 again becomes de-energized, and the locking spring 307 again applies the locking mechanism 308 to the tension member 120. During diastolic filling, the locking mechanism 308 prevents the energy transfer mechanism 306 from applying tension to the tension member 120. Otherwise, the tension applied to the tension member 120 by the energy transfer mechanism 306 would overcome the chamber pressure and reposition the restraining bars 111 to a closely spaced relationship during diastolic filling.

In one embodiment of the present invention, the solenoid 309 is energized by a voltage potential stored in a capacitor 303 having a capacitance (Cₚ). The capacitor 303 is charged by an inductive coil 305 when a magnet 310 attached to the tension member 120 is drawn through the inductive coil 305 during repositioning of the restraining bars 111. The current generated by the inductive coil 305 passes through a rectifier 304 so that the capacitor 303 is charged regardless of the direction in which the magnet 310 moves, capturing energy from repositioning the restraining bars 111 in both directions. Alternatively, the power for charging capacitor 303, or for directly energizing the solenoid, may be provided by a power source, such as a sub-cutaneous battery (not shown) or other power device known in the art.

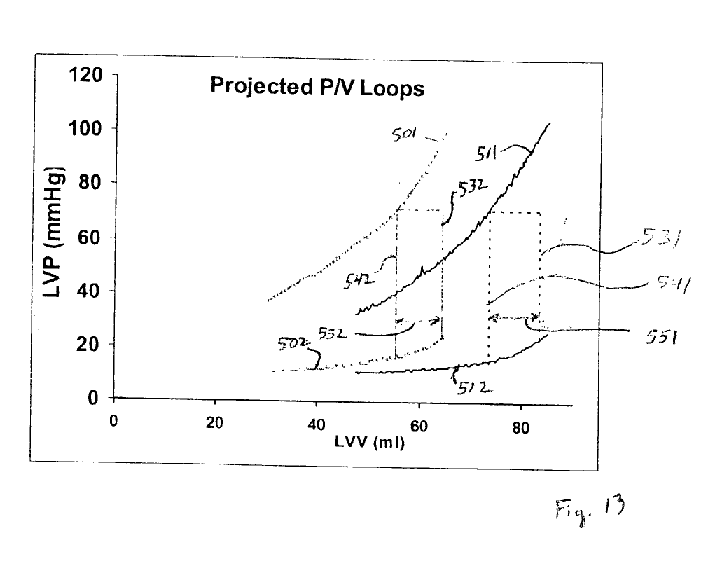

Referring now to FIG. 11, the control system of FIG. 10 is shown disposed on a clasp 110 according to an embodiment of the present invention. A highlighted connecting segment 112A is joined at each end to another connecting segment 112 by a hinge 113. The tension member 120 passes through a lumen at either end of the connecting segment 112A and passes either on the outer surface of connecting segment 112A (as shown) or internal to segment 112A (not shown). The tension member 120 passes through the induction coils 305 disposed on connecting segment 112A. Magnets (not shown) are attached to the tension member 120 where it passes through the induction coils 305. Each induction coil 305 is electrically connected to a rectifier 304. The rectifiers 304 are then electrically connected to the capacitor 303, so that when the tension member 120 moves relative to the induction coils 305 during repositioning of the restraining bars 111 (not shown in FIG. 11), the capacitor 303 is charged.

The capacitor 303 is electrically connected to the relay switch 302. The relay switch 302 is also electrically connected to a programmable electronic circuit 301 (not shown in FIG. 11) via the electrical leads 311 and to the solenoid 309. When the relay switch 302 receives a signal from the electronic circuit 301 via the leads 311, it closes the circuit discharging capacitor 303 and the energizing solenoid 309. When energized, the solenoid 309 overcomes the locking spring 307, releasing the locking mechanism 308 and allowing the energy transfer mechanism 306 to balance with the internal pressure of a chamber of the heart 10 through the restraining bars 111 and the tension member 120. It should be noted that the system illustrated in FIG. 11 differs from the system illustrated in FIG. 10 in that the energy transfer mechanism 306 acts on the tension member 120 through the locking mechanism 308 in FIG. 11 as opposed to acting directly on the tension member 120 in FIG. 10.

FIG. 12 shows the operation of the clasp 110 applied to a left ventricle 12 according to one embodiment of the present invention as a function of time with the electrical rhythm of the natural heart 10 as measured by an electrograph (EKG) 401 and the internal pressure of the left ventricle (LVP) 402 of the natural heart 10 superimposed on the horizontal time axis. The time axis arbitrarily begins prior to systolic ejection. At step 1, the EKG 401 is at its baseline and LVP 402 is low. The restraining bars 111 are distantly spaced and the left ventricle 12 is not restructured. The locking mechanism 308 is locked, keeping stored energy in the energy transfer mechanism 306.

In step 2, EKG 401 produces an R wave and the adapted programmable electronic circuit 301 (not shown in FIG. 12) provides a trigger signal 403 to the relay switch 302 (not shown in FIG. 12) to energize the solenoid 309 (not shown in FIG. 12) unlocking the locking mechanism 308. The energy stored in the energy transfer mechanism 306 overcomes the LVP 402, which is at about 5 to 30 mmHg, and the tension member 120 (not shown in FIG. 12) closes the hinged joints 114 (not shown in FIG. 12) repositioning restraining bars 111 into a closely spaced relationship and restructuring left ventricle 12.

The solenoid 309 only remains energized for a very short time, following which the locking spring 307 (not shown in FIG. 12) locks the locking mechanism 308. The solenoid 309 remains energized for a sufficient period of time to allow repositioning to occur, typically less than 200 milliseconds and preferably less than 25 milliseconds. Most preferably, there is a 0-20 millisecond delay.

During step 3, the locking mechanism 308 remains locked and the energy transfer mechanism 306 does not store energy. The LVP 402 increases during systolic ejection. The restraining bars 111 remain in a closely spaced relationship, because the locking mechanism 308 is locked, preventing the increasing LVP 402 from overcoming the tension in the energy transfer mechanism 306. Accordingly, the left ventricle 12 remains in a restructured state, reducing wall stress during systolic ejection.

In step 4, the electronic circuit 301 produces a delayed R-wave trigger signal 404, energizing the solenoid 309 and unlocking the locking mechanism 308. The delay is timed to unlock the locking mechanism 308 during at least very late systolic ejection or at the start of isovolumic relaxation but preferably during isovolumic relaxation. The delay is therefore, approximately 40 percent of the RR interval (the period between successive R-waves). The LVP 402, which is now about 50 to 120 mmHg, overcomes the energy transfer mechanism 306 and the restraining bars 111 are repositioned to a distantly spaced relationship while the energy transfer mechanism 306 stores energy to balance the tension forces in the tension member 120. With the restraining bars 111 repositioned to a distantly spaced relationship, the left ventricle 12 is able to return to an essentially unrestricted state.

The solenoid 309 only remains energized for a very short time, following which the locking spring 307 locks the locking mechanism 308. Step 1 is repeated with the locking mechanism 308 locked, and the energy transfer mechanism 306 storing energy. With the LVP 402 low during diastole, the stored energy in the energy transfer mechanism 306 is prevented from overcoming the LVP 402 to reposition the restraining bars 111 to a closely spaced relationship by locked locking mechanism 308. Instead, the restraining bars 111 remain in a distantly spaced relationship until the locking mechanism 308 is unlocked again by an R-wave-induced signal 403 in step B. As shown in FIG. 12, the natural heart 10 and the clasp 110 continue to cycle through the four steps (1-4) described above.

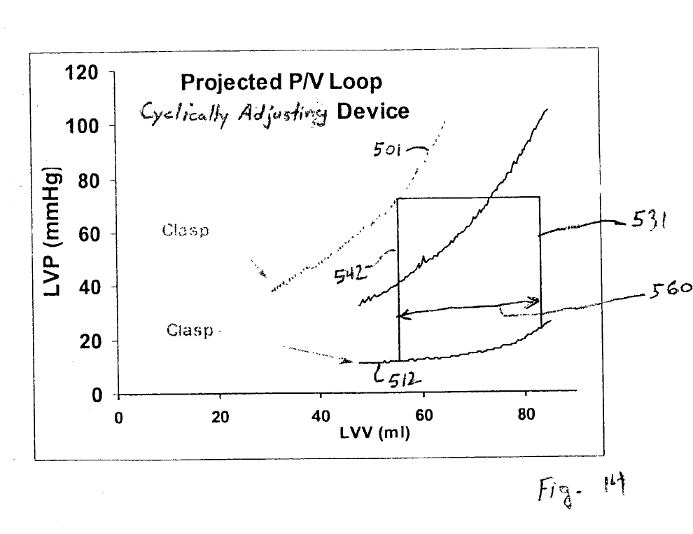

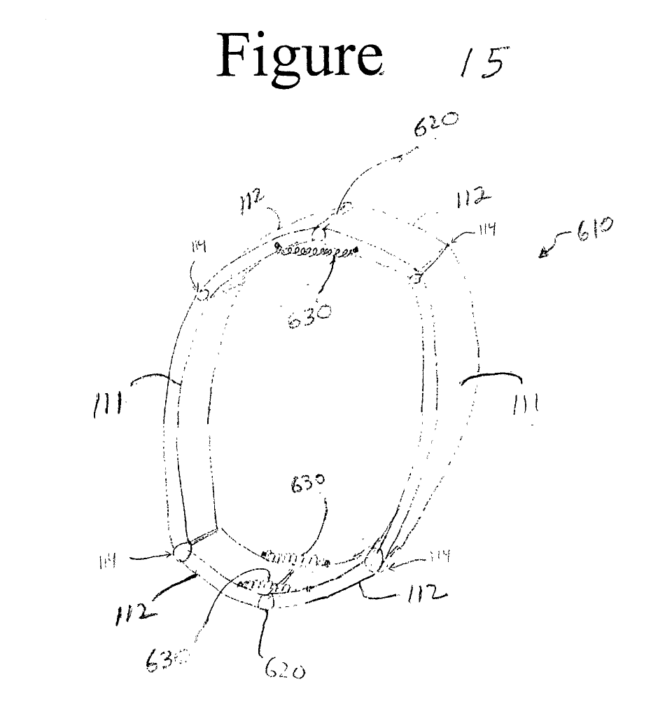

FIGS. 13 and 14 show the consequences of this cyclic clasp on ventricular performance. These figures were derived from an isolated canine heart failure preparation. FIG. 13 is a comparative diagram of left ventricle pressure (LVP) versus volume (LVV) for a natural canine heart showing experimentally derived End Systole Pressure (ESP) and End Diastole Pressure (EDP) curves and the Pressure-Volume (PV) cycle for each of: (1) a left ventricle 12 with a clasp providing continuous geometric reshaping or reconfiguration (clasp-restraining) and (2) an unrestricted left ventricle (baseline—no clasp or minimally restraining clasp). The clasp-restraining ESP curve 501 shows a large upward shift in ESP versus volume compared to the baseline ESP curve 511. This is a positive systolic effect corresponding to reduced wall stress. The clasp can have a negative effect, however, on diastolic function. The clasp-restraining EDP 502 curve is also shifted upward and to the left compared to the baseline EDP curve 512. This shift corresponds to a greater pressure requirement for diastolic filling with a clasp 110 on the left ventricle 12 than with an unrestricted left ventricle 12.

Also shown in FIG. 13 are projected PV cycles (loops). The PV cycles can be used to estimate the combined systolic and diastolic effects on the stroke volume of the left ventricle 12 (i.e., the volume of blood pumped in a single compression or beat). The sample volume data below are characteristic of a heart much smaller than that of an adult human and would be representative of a child or of a small animal. The exemplary baseline PV cycle assumes a filling pressure of 23.7 mmHg (the internal pressure on the left ventricle during diastolic filling). The baseline end diastolic volume 531 is 83.4 ml. Assuming that the left ventricle reaches an end systolic pressure of 73 mmHg during ejection, the baseline end systolic volume 541 would be 73.4 ml (the point where the baseline ESP curve 511 intersects 73 mmHg). The difference between baseline end diastolic volume 541 and baseline end systolic volume 531 is the baseline stroke volume 551. In the example illustrated in FIG. 13, the baseline stroke volume 551 is 10 ml. Again using the assumptions of a filling pressure of 23.7 mmHg and an end systolic pressure of 73 mmHg, the clasp-restraining end diastolic volume 532 would be only 64.4 ml and the clasp-restraining end systolic volume 542 would be 55.4 ml. Therefore, the clasp-restraining stroke volume 552 for the example of FIG. 13 would be only 9 ml.

As shown in FIG. 13, as the clasp decrease one dimension of the left ventricle, the end-systolic (501) and end-diastolic (512) pressure-volume relationships are shifted upward and to the left. The magnitude of this shift is depended upon the magnitude of the decrease in the left ventricular dimension caused by the clasp: the greater the decrease in dimension, the greater the shifts in the pressure-volume relationships. As described before, the magnitude of the clasp induced decrease in left ventricular dimension is limited by the negative effect on diastolic function. The cyclic clasp, as shown in FIG. 14, reduces or eliminates this impediment to systolic reconfiguration. Thus, the cyclic clasp can cause greater decreases in the left ventricular dimension (of up to 40%, 60%, or even more) while maintaining effective diastolic function.

FIG. 14 is a left ventricle pressure versus volume diagram for a natural canine heart 10 showing a calculated PV cycle for a cyclical clasp 110 according to the exemplary embodiment of the present invention described with respect to FIGS. 5-8 and 10-12. The filling pressure is again assumed to be 23.7 mmHg. Prior to diastolic filling, the clasp 110 is allowed to reposition to a distantly spaced relationship approximately following the baseline EDP curve 512. Therefore, the end diastolic volume is approximately the baseline end diastolic volume 531 of 83.4 ml. Prior to systolic ejection, restraining bars 111 are repositioned inwardly to a closely spaced relationship causing the left ventricle 12 to shift to the clasp-restraining ESP curve 501. Therefore, the end systolic volume is approximately the clasp-restraining end systolic volume 542 of 55.4 ml. Accordingly, the cyclical clasp stroke volume 560 is 28 ml, representing a significant improvement in performance.

B. Alternate Cyclical Clasp

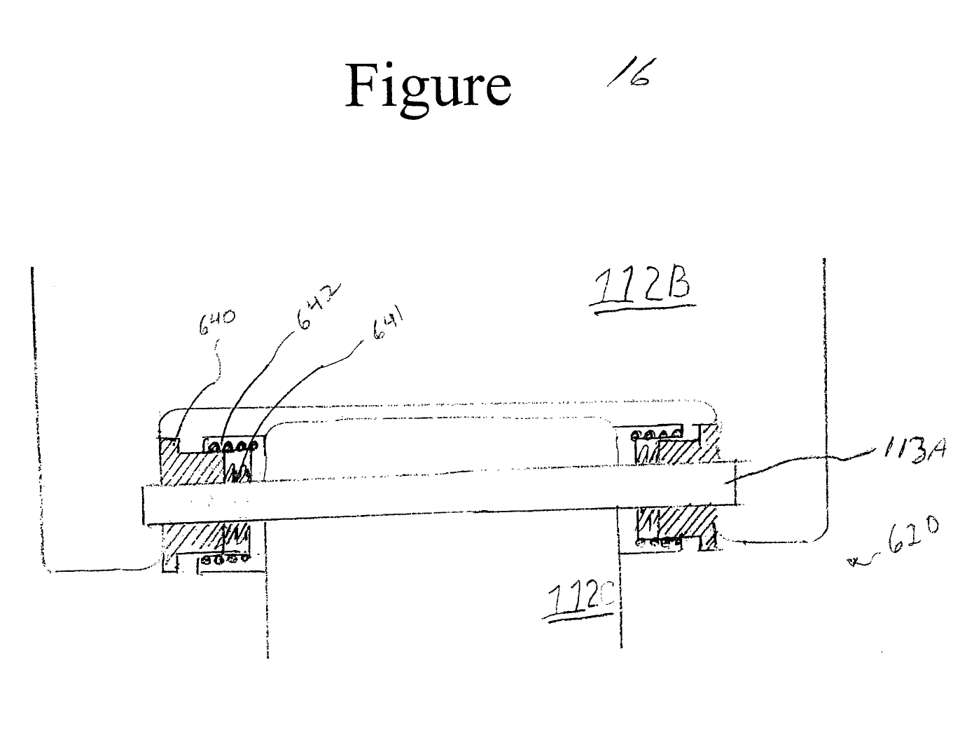

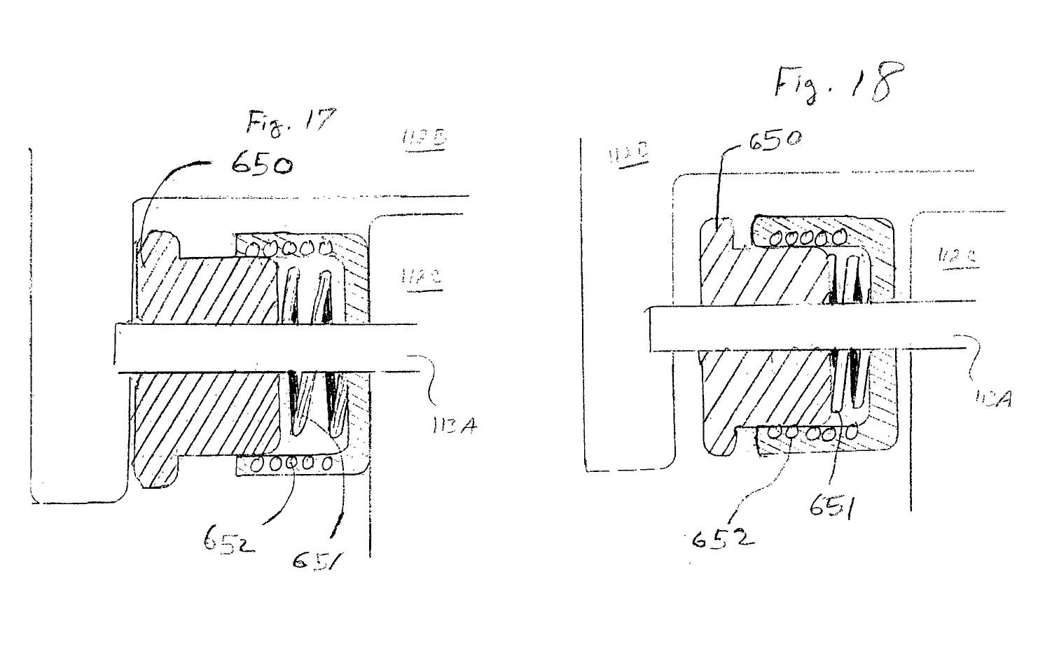

FIG. 15 illustrates an alternate cyclical clasp 610. Alternate cyclical clasp 610 comprises two restraining bars 111 connected together at both ends by a pair of connecting segments 112. Each connecting segment 112 is connected at one end to a restraining bar 111 by a hinged joint 114. Each pair of connecting segments 112 is connected together by a center hinged joint 620. As shown in FIG. 15, cyclical energy transfer mechanisms 630 (e.g., springs) are disposed across each center hinged joint 620 biasing the restraining bars 111 inwardly. Alternate cyclical clasp 610 is disposed around a chamber of a natural heart 10, such as a left ventricle 12.

At the beginning of systole, the cyclical energy transfer mechanisms 630 overcome the internal pressure of the left ventricle 12 and reposition the restraining bars 111 inwardly to a closely spaced relationship. When the restraining bars 111 are in a closely spaced relationship, the chamber is reconfigured as contiguous portions of truncated ellipsoids and wall stress is reduced. During isovolumic relaxation, the internal pressure in the chamber overcomes the force of the cyclical energy transfer mechanisms 630, repositioning the restraining bars 111 to a distantly spaced relationship. As described above, the step of allowing the restraining bars 111 to be repositioned during diastole enhances diastolic filling.

FIG. 16 shows an exemplary embodiment of center hinge joint 620 in greater detail with two adjacent segments 112C, 122B comprising respectively a tongue and a groove rotatably connected by a hinge pin 113A. As shown in FIG. 16, the alternate cyclical clasp 610 comprises a locking mechanism 640 mounted on a hinge pin 113A in the center hinged joints 620. The locking mechanism 640 is prevented from rotating on the hinge pin 113A, for example, by a key and slot. The hinge pin 113A is prevented from rotating relative to a first connecting segment 112C but a second connecting segment 112B is able to rotate relative to the hinge pin 113A when the locking mechanism 640 is unlocked. A locking spring 641 is provided for each locking mechanism 640, biasing the locking mechanism 640 into contact with the second connecting segment 112B and preventing the second connecting segment 112B from rotating relative to the hinge pin 113A and repositioning the restraining bars 111 (not shown in FIG. 16). A solenoid 642 overcomes the locking spring 641 when energized, unlocking the locking mechanism 640. The locking mechanisms 640 are shown in a locked position in FIG. 16.

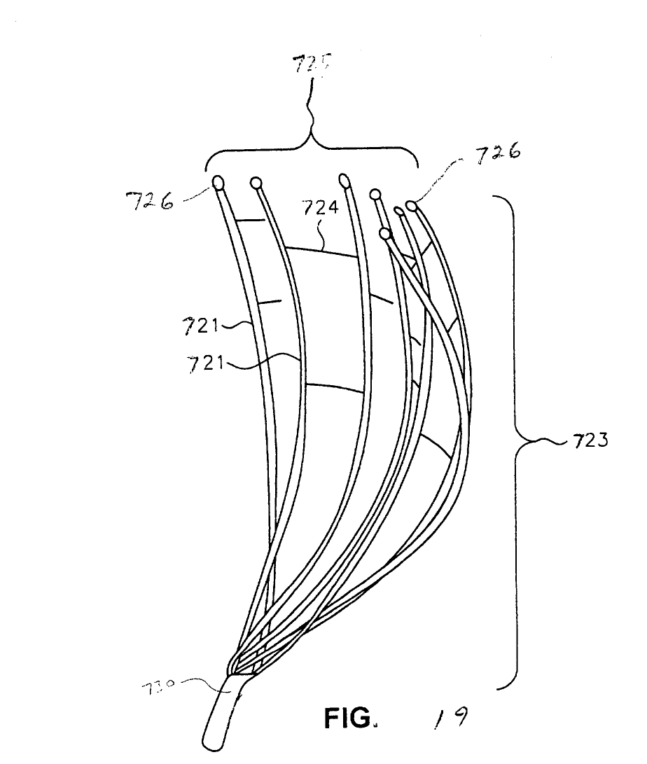

FIGS. 17 and 18 show a locking mechanism 650 and solenoid 652 that are mounted on hinge pin 113A between first connecting segment 112C and second connecting segment 112B. When the solenoid 652 is not energized, a locking spring 651 forces the locking mechanism 650 and the solenoid 652 apart, such that one of the locking mechanism 650 or the solenoid 652 is pressed against the first connecting segment 112C and the other one of the locking mechanism 650 and the solenoid 652 is pressed against the second connecting segment 112B. The simultaneous pressure against the first connecting segment 112C and the second connecting segment 112B prevents relative rotation of the connecting segments 112B, 112C and consequently repositioning of the restraining bars 111 (not shown in FIG. 17 or 18). In FIG. 17, the locking mechanism 650 is locked preventing the restraining bars 111 from repositioning. In FIG. 18, the locking mechanism 650 is unlocked allowing the restraining bars 111 to be repositioned.

The solenoids 642, 652 can be energized by any of a number of methods. For example, a battery and circuit can be used to energize solenoid 642, 652 according to electrical pulses generated by the natural heart 10. Alternatively, an energy-recovery circuit can be provided on one or more moving parts of the cyclical clasp 110, 610. As can be appreciated by those skilled in the art, the locking mechanism 640, 650 comprises a conductive material such that it is displaced by a magnetic field created when the solenoid 642, 652 is energized.

C. Pressure-Transfer Mechanism

In an alternative embodiment of the present invention, an alternative energy transfer mechanism is used in conjunction with a clasp to apply cyclical forces to a chamber wall of a natural heart 10. FIG. 19 shows a pressure-transfer mechanism 723 consisting of a structure configured to be positioned inside a chamber of a natural heart 10 such that it absorbs energy in a first state during systolic ejection and releases the stored energy to enhance diastolic filling. The pressure-transfer mechanism 723 may comprise a plurality of spring elements 721 arranged in a fan-shaped array 725 and typically disposed adjacent an interior surface of a ventricular wall. The pressure-transfer mechanism 723 may further comprise an apical end 730 joining the individual spring elements 721 to form a spring bundle shaped and configured to extend through the apical portion 20 of a natural heart 10. Spring elements 721 may be connected by tethers 724 to maintain a preferred spacing between the spring elements 721. Springs tips 726 may be provided at the ends of spring elements 721 to prevent damage to heart tissue.

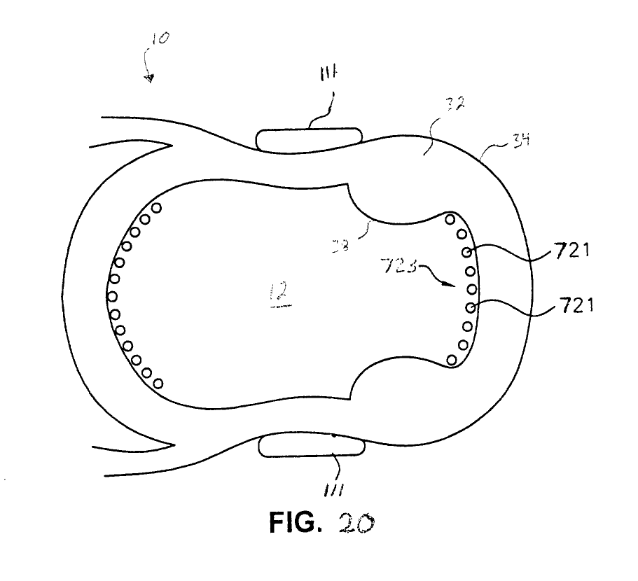

FIG. 20 is a partial horizontal cross-sectional view of a natural heart 10 showing a chamber (e.g., left ventricle 12) which is reconfigured to reduce wall stress by restraining bars 111 in a fixed closely spaced relationship and the function of which is enhanced during diastolic filling by the pressure-transfer mechanism 723. Throughout a cardiac cycle, the pressure-transfer mechanism 723 applies an outward force to interior (endocardial) surface 38 of heart chamber 12. The outward force is inversely proportional to the chamber radius. Also shown in FIG. 20 are the spring elements 721 of the pressure-transfer mechanism 723 and the cardiac tissue 32 and the epicardial surface 34 of the heart 10.

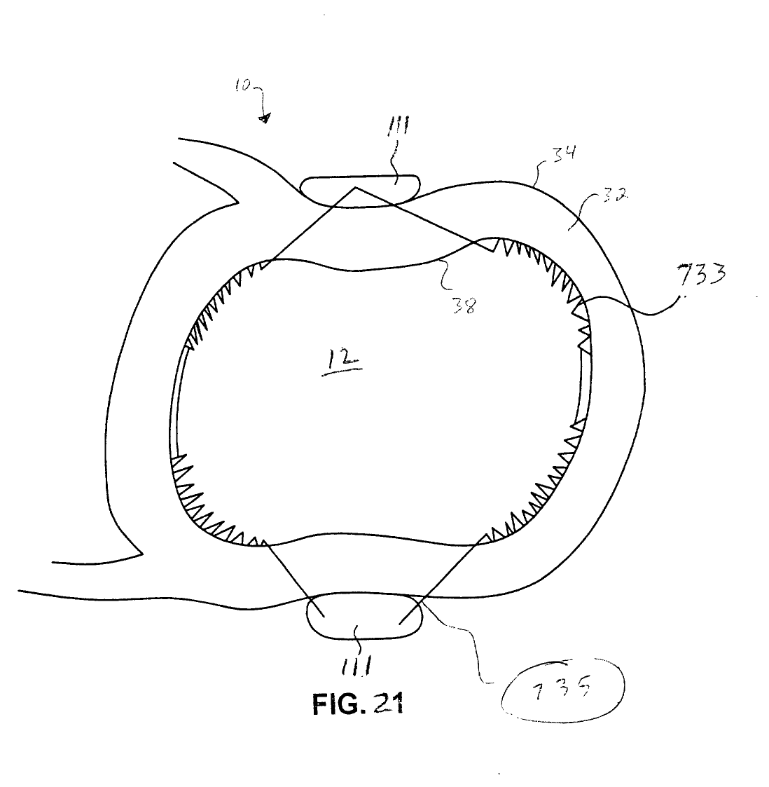

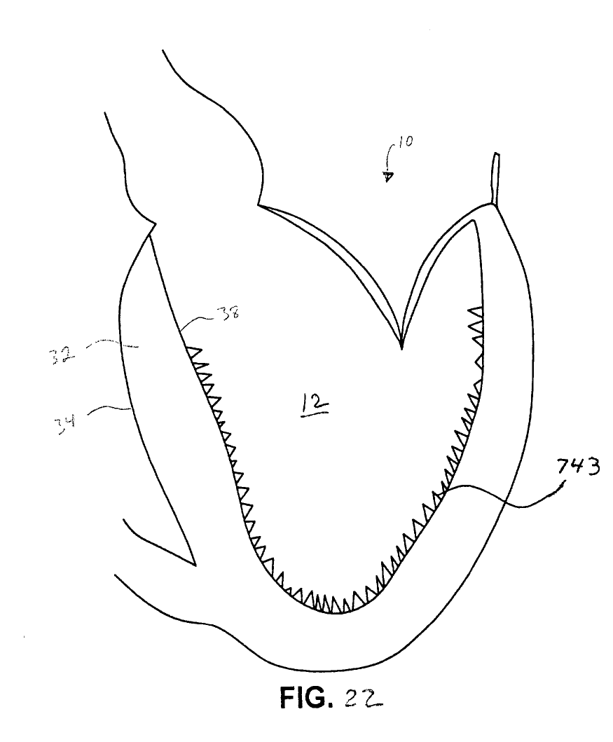

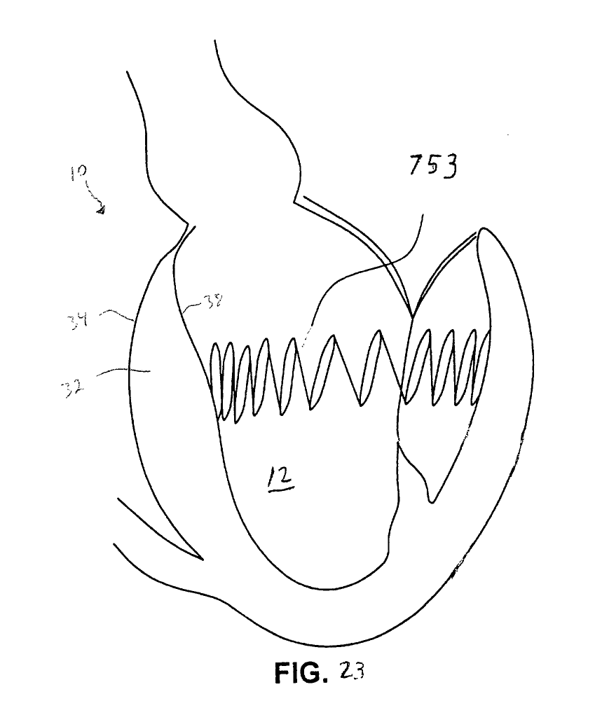

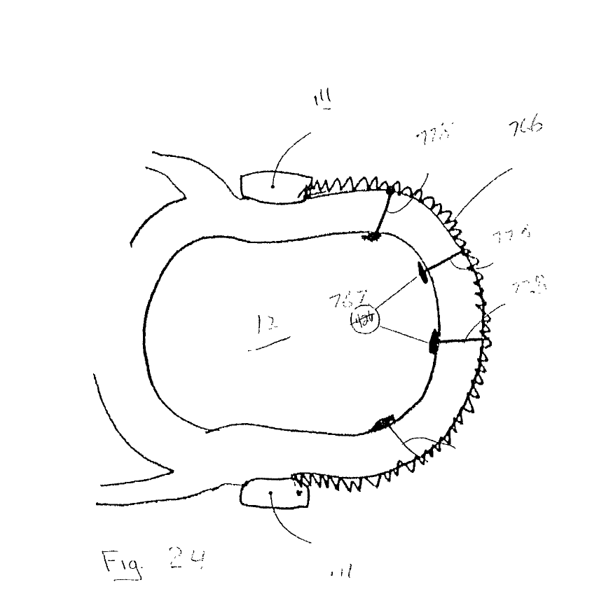

Alternatively, a pressure-transfer mechanism 733 may comprise one or more compression springs disposed horizontally adjacent the endocardial surface 38 of a chamber (e.g., left ventricle 12) as shown in FIG. 21. The pressure-transfer mechanism 733 may optionally be connected to the restraining bars 111 through the chamber wall using a connector 735 as shown in FIG. 21. FIG. 22 shows another alternative pressure-transfer mechanism 743 comprising a compression spring disposed vertically adjacent the endocardial surface 38 of the left ventricle 12. FIG. 23 shows yet another alternative pressure-transfer mechanism 753 comprising a compression spring disposed transverse the left ventricle 12. FIG. 24 shows still another alternative pressure-transfer mechanism 763 comprising a spring 766 disposed external the left ventricle 12 tied to pads 767 adjacent the endocardial surface of left ventricle 12 by connectors 778 extending through the chamber wall. Spring 766 may be connected to restraining bars 111, as shown.

In operation, the pressure-transfer mechanism 723, 733, 743, 753, 763 absorbs and stores energy during systolic ejection. The contraction force during systolic ejection overcomes the spring force of the pressure-transfer mechanism 723, 733, 743, 753 and the chamber wall moves inwardly, imparting energy into the springs. Following systole, the heart 10 relaxes allowing the left ventricle 12 to expand during diastolic filling. The pressure-transfer mechanism 723, 733, 743, 753 releases stored energy, enhancing the chamber expansion and diastolic filling.

Having shown and described the preferred embodiments to the present invention, further adaptations of the cyclical clasp for the living heart as described can be accomplished by appropriate modifications by one of ordinary skill in the art without departing from the scope of the present invention. For example, the present invention can be used with any one or even with a plurality of the various chambers of a living heart, and also could be used with different structural embodiments to restructure the chamber. Several such potential modifications have been discussed and others will be apparent to those skilled in the art. Accordingly, the scope of the present invention should be considered in terms of the following claims and is understood not to be limited in the details, structure, and operation shown and described in this specification and drawing.

What is claimed:

1. A device for treating a natural heart having a plurality of chambers and a cardiac cycle comprising isovolumic contraction, systolic ejection, isovolumic relaxation, and diastolic filing, the device comprising:
a plurality of members adapted to inwardly displace portions of a wall of one chamber of the natural heart; and
a connector adapted to maintain the members in a closely spaced relationship to reconfigure a chamber of the natural heart during at least a portion of systolic ejection;
wherein cyclical forces are applied to the wall of the one chamber of the natural heart to reduce adverse effects caused by reconfiguring the chamber during diastolic filling.

2. The device of claim 1 wherein the plurality of members comprises two members spaced approximately 180 degrees apart, which members are adapted to inwardly displace underlying portions of a wall of one chamber of the natural heart.

3. The device of claim 1 wherein the cyclical forces are applied by the members adapted to inwardly displace portions of a wall of one chamber of the natural heart during at least a portion of the cardiac cycle.

4. The device of claim 3 wherein the connector comprises a plurality of connecting segments joining together the members through a series of hinged joints.

5. The device of claim 4 further comprising an energy transfer mechanism that stores energy expended by the natural heart during one portion of the cardiac cycle and releases energy to reposition the members such that the chamber is substantially more restricted during another portion of the cardiac cycle.

6. The device of claim 5 further comprising a locking mechanism preventing the energy transfer mechanism from storing energy during at least most of systolic ejection and preventing the energy transfer mechanism from releasing energy during at least most of diastolic filling.

7. The device of claim 6 wherein the locking mechanism is unlocked during late systole or during isovolumic relaxation of the cardiac cycle.

8. The device of claim 7 wherein the locking mechanism is unlocked using energy captured from metabolic functioning of the heart.

9. The device of claim 7 wherein the locking mechanism is unlocked using energy from a battery or other exogenous source.

10. The device of claim 3, the plurality of members comprising first and second members, wherein said first and second members are positioned adjacent the epicardial surface of the chamber wall in a spaced relationship relative to each other about 180 degrees apart.

11. The device of claim 3, the plurality of members comprising a first member configured to be positioned adjacent the anterolateral surface of the chamber and a second member configured to be positioned adjacent the posteromedial surface of the chamber.

12. The device of claim 3, comprising at least one fixation device configured to maintain contact between the members and a specific surface of the chamber.

13. The device of claim 1 wherein the cyclical forces are applied outwardly to the endocardial surface of the chamber wall by a pressure-transfer mechanism adapted to be placed inside the chamber.

14. The device of claim 13 wherein the pressure-transfer mechanism comprises one or more springs.

15. The device of claim 13 wherein the pressure-transfer mechanism is fixedly attached to the connector.

16. The device of claim 13 wherein the pressure-transfer mechanism is fixedly attached to the members.

17. A method of treating a natural heart having a plurality of chambers and a cardiac cycle comprising isovolumic contraction, systolic ejection, isovolumic relaxation, and diastolic filing, the method comprising:
inwardly displacing portions of a wall of one chamber during at least a portion of a cardiac cycle; and
applying a cyclical force to the wall of the one chamber to reduce adverse effects on diastolic filling caused by displacing portions of the wall.

18. The method of claim 17 wherein the wall is inwardly displaced by restraining bars during at least most of systolic ejection.

19. The method of claim 18 wherein the cyclical force is applied by the restraining bars to reposition the restraining bars to a closely spaced relationship prior to systolic ejection and the restraining bars are positioned in a distantly spaced relationship applying a lesser force during at least most of diastolic filling.

20. The method of claim 19 wherein the restraining bars are repositioned into a closely spaced relationship prior to systolic ejection by an energy transfer mechanism.

21. The method of claim 19 wherein the restraining bars are allowed to reposition into a distantly spaced relationship prior to diastolic filling.

22. The method of claim 19 wherein the restraining bars are repositioned by balancing pressure inside the chamber with energy stored in an energy transfer mechanism, and the restraining bars are prevented from repositioning by a locking mechanism which prevents balancing the pressure inside the chamber with energy stored in the energy transfer mechanism.

23. A device for treating a natural heart, comprising:
a plurality of members pressing inwardly on a chamber wall of the heart to reconfigure the chamber into at least two contiguous communicating portions of truncated ellipsoids during at least most of systolic ejection; and
an energy transfer mechanism configured to reduce adverse effects of reconfiguration during at least most of diastolic filling.

24. A cyclical device for treating a natural heart, comprising:
a plurality of members configured to be positioned adjacent the epicardial surface of a chamber of the heart; and
a connector fixing the members in a first spaced relationship during at least most of systolic ejection and allowing the heart to move the members to a second spaced relationship prior to diastolic filling,
wherein the second spaced relationship provides a greater chamber radius than the first spaced relationship.

25. The device of claim 24 further comprising at least one structure disposed adjacent an interior surface of the chamber and adapted to exert an outward force thereon during at least most of diastolic filling.

26. The device of claim 25 wherein the structure comprises an array of spring elements.

27. A device for treating a natural heart, comprising:
a plurality of members configured to inwardly displace portions of a wall of a chamber of the heart; and
a pressure-transfer mechanism configured to provide an outward force on the wall of the chamber, wherein the outward force decreases as the chamber expands.

28. A clasp configured to be placed about a chamber of a natural heart for treatment of the natural heart, the clasp comprising:
a plurality of restraining bars adapted to be placed adjacent a chamber wall external to the chamber and to reconfigure the chamber as contiguous truncated portions of ellipsoids when positioned in a closely spaced relationship to each other;
a connecting component adapted to allow the restraining bars to be positioned in either of a closely spaced relationship and a distantly spaced relationship;
an energy-storage member adapted to reposition the restraining bars from the distantly spaced relationship to the closely spaced relationship; and
a locking mechanism preventing the restraining bars from repositioning during at least one portion of a cardiac cycle.
